(19) United States
(12) Patent Application Publication
(10) Pub. No.: US 2012/0301951 A1
(43) Pub. Date: Nov. 29, 2012

(54) PREVENTION OF CONTAMINATION OF NUTRIENT FEED RESERVOIRS & FEED LINES IN BIOREACTOR SYSTEMS

(75) Inventor: SCOTT CHRISTOPHER JACKSON, Wilmington, DE (US)

(73) Assignee: E. I. DU PONT DE NEMOURS AND COMPANY, Wilmington, DE (US)

(21) Appl. No.: 13/297,552

(22) Filed: Nov. 16, 2011

Related U.S. Application Data
(60) Provisional application No. 61/414,917, filed on Nov. 18, 2010.

(51) Int. Cl. C12N 1/20; C12M 1/00

(52) U.S. Cl. 435/253.3; 435/252.1; 435/289.1

(57) ABSTRACT

A method to prevent contamination of feed line(s) and nutrient feed reservoirs(s) is disclosed. In this method the growth medium which flows from the nutrient feed reservoir through the feed line to the bioreactor contains at least one nutritional component supplied at a concentration which is inhibitory to cell growth. The inhibitory nutritional component in the growth medium prevents back growth of cells from the bioreactor into the feed line(s). The growth medium with inhibitory nutritional component is diluted prior to or at entering the bioreactor.

This application claims the benefit of U.S. Provisional Application 61/414,917, filed Nov. 18, 2010, and is incorporated by reference in its entirety.

### FIELD OF THE INVENTION

The current method relates to the field of microbiology and contamination prevention. Specifically, it relates to a method for prevention of contamination of nutrient feed reservoirs and feed lines providing nutritional components to bioreactors.

### BACKGROUND OF THE INVENTION

Bioreactor systems are commonly used for growth and production of microbial, mammalian and plant cells for various industrial and pharmacological applications. One of the universal problems in operating bioreactor systems, which surpasses mechanical, electrical or instrumentation problems, is the contamination problem which results in process failure. Contamination prevention in bioreactor systems is therefore important to allow uninterrupted production of the desired cells and/or products and to meet manufacturing and production timelines. Additionally, interruption of contaminated bioreactors, clean-ups and start-ups are costly and can have significant economic impact on the entire process (Junker, B., et al., J. Biosc. Bioeng., 102: 251-268, 2006).

Various methods have been used to prevent contamination or spread thereof. Some of these methods depend on application of technologies detrimental to cell growth such as using lethal temperatures, ultraviolet radiation, ionizing radiation, and adding chemical inhibitors to the growth medium. For example:

U.S. Pat. No. 4,192,988 disclosed application of electrical heating for heating a sink drain barrier to prevent growth of microorganisms in the drain.

U.S. Pat. No. 3,985,994 disclosed application of an electric heater to heat the interior of the connecting pipe portion between the outlet pipe and the drain pipe of a wash basin to prevent microbes from rising from the drain pipe into the outlet pipe.

Application of ultraviolet light for contamination control in blood products was disclosed in WO01/744071 and U.S. Patent Publication 2008/0142452 disclosed use of UV light in killing microorganisms during water treatment.

Application of intervening physical devices such as air-breaks and filters that physically interrupt penetration of cells and thus prevent contamination has been known and commonly practiced by those knowledgeable in the art (Stanbury, P. R. et al., Principles of Fermentation Technology, 2^{nd} Edition. 1995, Elsevier Sciences Limited, Burlington, Mass.). In the commonly owned application publication WO 2004101479 filtration was used for removing microbial cells from the product stream of the bioreactor.

Application of bioreactor systems for production of various industrial chemicals and pharmaceutical products, and other applications, has been increasing over the past two decades, thus there is a need for developing effective and economical methods to prevent contamination in these systems without application of intervening physical devices and/or harsh chemicals.

### SUMMARY OF THE INVENTION

The method disclosed herein addresses the need for preventing contamination in a bioreactor system. Specifically, the method teaches prevention of contamination of nutrient feed reservoir(s) and feed line(s) in a bioreactor system during its operation. In the currently disclosed method, no intervening physical device in either the nutrient feed reservoir or the feed line is used for contamination prevention. Rather, contamination prevention is effected through using a growth medium containing one or more inhibitory nutritional components. The bioreactor system disclosed herein comprises a bioreactor, a brine feed reservoir, at least one nutrient feed reservoir, and at least first, second, and third feed lines that connect the feed reservoirs to the bioreactor. In the disclosed method the growth medium which flows through the second feed line to the bioreactor contains at least one nutritional component supplied at a concentration which is inhibitory to cell growth.

In an aspect, the disclosed method for preventing contamination of the feed lines and feed reservoirs in a bioreactor system, wherein the bioreactor system comprises a bioreactor, a brine feed reservoir, at least one nutrient feed reservoir, and at least first, second, and third feed lines that connect the feed reservoirs to the bioreactor, comprises the steps of:

a) providing the bioreactor with an initial charge of cells and inoculum medium;

b) filling the first feed line with brine solution from the brine feed reservoir;

c) filling the second feed line with a second growth medium, comprising one or more inhibitory nutritional component, from the

nutrient feed reservoir; and

d) feeding the brine solution in the first feed line of (b) and the second growth medium in the second feed line of (c) into the third feed line where they combine forming a growth medium mixture; whereby the inhibitory component of the second growth medium is diluted with brine solution in the growth medium mixture before or at the point of entering the bioreactor.

In another embodiment the invention provides a bioreactor system comprising a bioreactor, a brine feed reservoir, at least one nutrient feed reservoir containing a second growth medium comprising one or more inhibitory nutritional component, and at least first, second, and third feed lines that connect the feed reservoirs to the bioreactor, wherein the first and second feed lines each connect to a reservoir at one end and to the third feed line at the other end.

### BRIEF DESCRIPTION OF THE FIGURE

FIG. 1 is a diagram of a bioreactor system with multiple feed lines.

### INFORMATION ON DEPOSITED STRAINS

Applicants have made the following biological deposits under the terms of the Budapest Treaty on the International Recognition of the Deposit of Microorganisms for the Purposes of Patent Procedure:

| | International | |
|---|---|---|
| Depositor Identification | Depository | |
| Reference | Designation | Date of Deposit |
| Pseudomonas stutzeri | ATCC No. PTA-11283 | Sep. 9, 2010 |
| BR5311 | | |
| Arcobacter sp 97AE3-12 | ATCC No. PTA-11409 | Oct. 14, 2010 |
| Arcobacter sp 97AE3-3 | ATCC No. PTA-11410 | Oct. 14, 2010 |
| Pseudomonas stutzeri | ATCC No. PTA-8822 | Dec. 4, 2007 |
| LH4:18 | | |
| Pseudomonas stutzeri | ATCC No. PTA-8823 | Dec. 4, 2007 |
| LH4:15 | | |
| Thauera aromatica strain | ATCC No. PTA-9497 | Sep. 17, 2008 |
| AL9:8 | | |

### DETAILED DESCRIPTION

This invention relates to preventing contamination during cell growth in a bioreactor system. More specifically it relates to a method that inhibits back growth of cells from the bioreactor into a feed line and thus prevents contamination of the feed line and a connected nutrient feed reservoir. The term “back growth” as used herein refers to the penetration of cells from the bioreactor back into a feed line. The term “bioreactor” as used herein refers to a container used to grow cells that produce one or more products for commercial use, which may be the grown cells. The term “feed line”, as used herein, refers to at least a portion of a supply line that carries feed or growth medium from a feed reservoir to the bioreactor. The feed line can comprise several portions that deliver different feed stocks from different sources for mixing and/or subsequent delivery to the bioreactor or can comprise one line that feeds directly from one feed reservoir source. The term “feed reservoir” as used herein refers to a storage container from which growth medium or other solution is drawn in order to supply the bioreactor. The term “back growth”, is defined as penetration of cells growing in the bioreactor into the feed line leading to the nutrient feed reservoir.

The present invention may be used for preventing contamination of bioreactor systems which use cells of microbial, plant or mammalian origin. The present invention is also particularly useful in bioreactor systems where the growth medium is continuously fed into the bioreactor.

### Apparatus of the Current Method

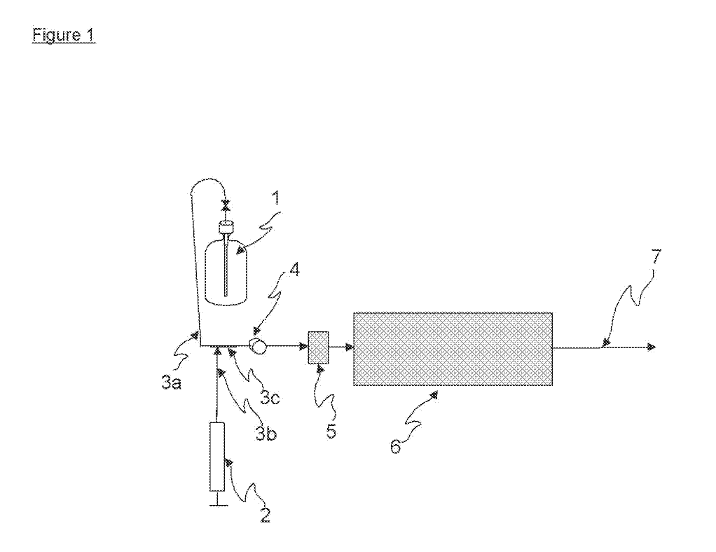

A bioreactor system useful in the present method comprises a bioreactor, multiple reservoirs, and multiple feed lines connecting said bioreactor and reservoirs. One embodiment of the bioreactor system is depicted in FIG. 1. In this system, a brine reservoir (1) is connected by a first feed line (3a) to a third feed line (3c) and further to a feed pump (4). A nutrient feed reservoir (syringe pump, 2) is connected via a second feed line (3b) to the third feed line (3c). The nutrient feed reservoir (2) can be connected to the third feed line (3c) either before or after the feed pump (4), or through the feed pump (4). Combined feed from the brine and nutrient feed reservoirs in the third feed line (3c) then goes into a mixing section (5) which can be a pump and/or a length of tubing and/or a static mixer and/or a mixing zone integral with the bioreactor (6) prior to entering the bioreactor. The location of the feed pump (4) and the mixing section (5) can be reversed as needed. Effluent from the bioreactor leaves via an exit line (7).

In the configuration shown in FIG. 1 the mixing section (5) mixes the feed from the nutrient feed reservoir with the feed from the brine reservoir in the third feed line (3c). The mixing of the two feeds continues during transfer to the bioreactor (6). The mixing section (5) can be located as close as possible to the bioreactor.

The nutrient feed reservoir (2) can be made of metal, glass, plastic or any other material that can be sterilized and that can maintain its sterility for any length of time. The bioreactor (6) is commonly used in fermentation industry and is well known to those skilled in the art (Chisti, Y., Chem. Eng. Proc., 88: 80-85, 1992 and Principles of Fermentation Technology, 2^{rd} Edition, 1995, Stanbury, P. F. ed., Elsevier, Burlington, Mass.). The bioreactor may be constructed of steel or glass or any other material suitable for the specific application in mind and can be built in various sizes. The temperature of the bioreactor is regulated by conventional methods, and can be maintained at the temperature optimal for growth of the cells used in the bioreactor. The feed lines that are pipe lines and/or tubing connecting the reservoirs to the bioreactor can be made of any material suitable for the specific application, which should withstand the pressure used during the process and can be sterilized. The dimensions of the feed lines vary depending on the application. The bioreactor system of the current method can be used for either fed-batch or continuous operations. The bioreactor system of the current method can be used for production of a variety of chemicals and/or products for industrial and/or pharmaceutical applications; said product may include cells grown in the bioreactor.

### Brine Reservoir

The brine reservoir (1) in the current method stores a brine solution. The concentration of salt in the brine solution may be up to 20 wt % (200 ppt), or more typically up to about 10 wt % (100 ppt). The term “salt” includes any ionic compound that can create ions in water including, but not limited to KCl, SrCl, NaBr, NaCl, CaCl₂, MgCl₂. The concentration of salt in a brine solution used in a specific system will depend on factors such as the brine solution feed rate and dilution with nutrient medium, and the tolerance or requirement for salt of the cells to be grown in the bioreactor, which may be readily determined by one skilled in the art. In one embodiment the brine solution contains 70 parts per thousand (ppt) salt and is obtained from a salty aquifer in Canada.

### Growth Media

Growth media useful in the present invention include at least one growth substrate (compounds that supply mass and energy for cell growth); and may include electron acceptors, nitrogen and phosphorus sources, as well as various trace elements such as vitamins and metals that are usually required, in addition to growth substrate and nitrogen sources, for cell growth and activity.

Nutritional components of the growth medium can include the following substances, alone or in combination: one or more carbon source added at greater than 20 parts per million (ppm): one or more electron acceptor for cell growth (under anaerobic growth conditions) added at greater than 50 ppm; a source of nitrogen added at greater than 1 ppm; a source of phosphorous added at greater than 1 ppm; a source of trace nutrients, such as vitamins and metals, added at greater than 1 ppm.

Useful nutritional components contemplated herein for the growth medium include those containing at least one of the following elements: C, H, O, P, N, S, Mg, Fe, or Ca. Non-limiting examples of such inorganic compounds include: PO₄²⁻, NH₄⁺, NO₂⁻, NO₂⁻, and SO₄²⁻ amongst others. In case of microbial cells, growth substrates can include sugars, organic acids, alcohols, proteins, polysaccharides, fats, hydrocarbons or other organic materials known in the art of microbiology to be subject to microbial decomposition. Nutritional components may include major nutrients containing nitrogen and phosphorus (non-limiting examples can include NaNO₃, KNO₃, NH₄NO₃, Na₂ HPO₄, K₂HPO₄, NH₄Cl); vitamins (non-limiting examples may include folic acid, ascorbic acid, and riboflavin); trace elements (non-limiting examples may include B, Zn, Cu, Co, Mg, Mn, Fe, Mo, W, Ni, and Se); buffers for environmental controls; catalysts, including enzymes; and both natural and artificial electron acceptors (non-limiting examples may include SO₄²⁻, NO₃⁻², Fe⁺³, humic acid, mineral oxides, quinone compounds, CO₂, O₂, and combinations thereof).

In inoculum growth medium, the medium components are those, such as described above, that support growth and productivity of the cells to be grown in the bioreactor, The nutrient components are present in concentrations that support growth of cells used in the bioreactor. One of skill in the art will know the components to use in the inoculum growth medium for a particular cell type.

In the current method, the second growth medium which is stored in the nutrient reservoir contains one or more of the above listed nutrient components in a high concentration such that it is inhibitory to cell growth, thus providing an inhibitory nutritional component. Typically high concentration will mean the use of any of these nutrients in excess of 10% wiv. For example many salts, sugars, esters, and alcohols are consumed for growth at low concentrations, but are inhibitory to growth at high concentrations (Microbial Ecology of Foods, V. 1, Silliker et al., (ed.) pages 70-158, 1980. Academic Press, New York, N.Y.). As used herein, “inhibitory nutritional component” means any nutrient in excess of 1 times the concentration at which the nutrient becomes inhibitory to the growth of cells used in the bioreactor. Alternatively, these nutrients can be used above 2 times the inhibitory nutrient feed concentration. In addition, these nutrients can be used above 3 times the inhibitory nutrient concentration or at their solubility limit in water at the nutrient feed temperature, whichever is lower. One of skill in the art will know, or can readily determine, the concentration at which a nutrient component becomes inhibitory to the growth of the cells to be used in the bioreactor.

In one embodiment the carbon source is acetate and in the second growth medium the acetate concentration is greater than 1% making acetate an inhibitory nutrient component. In another embodiment the carbon source is lactate and in the second growth medium the lacate concentration is greater than 1% making acetate an inhibitory nutrient component. In yet another embodiment the electron acceptor is nitrate and in the second growth medium the nitrate concentration is greater than 1% making nitrate an inhibitory nutrient component. Any combination of these components, or combinations with other components, may be used as inhibitory nutritional components.

### Feeds to the Bioreactor

In the present method, cells and inoculum growth medium are added as an initial charge to the bioreactor.

In the present method the feed line connected to the brine reservoir (first feed line) is filled with brine solution from the brine reservoir. The feed line connected to the nutrient feed reservoir (second feed line) is filled with the second growth medium, comprising one or more inhibitory nutritional component, from the nutrient feed reservoir. No intervening physical device in either the nutrient feed reservoir or the second feed line is used to prevent contamination. The presence of the inhibitory nutritional component in the second growth medium prevents contamination of the second feed line and the nutrient feed reservoir. The concentrations of components of the second growth medium that is stored in the nutrient feed reservoir remain constant in the absence of contamination.

The first and second feed lines connect to a third feed line such that the brine solution and second growth medium both feed into the third feed line where they combine forming a growth medium mixture. In the growth medium mixture the salt concentration of the brine solution is diluted, and the concentration of the inhibitory nutritional component is diluted. In one embodiment the inhibitory nutritional component is diluted in the growth medium mixture to less than about 10% of the concentration in the second growth medium. The inhibitory nutrient may be reduced to a level that is less than about 10%, 9%, 8%, 7% 6%, 5%, 4%, 3%, 2%, 1%, or less of the concentration of the inhibitory nutrient in the second growth medium. However, the inhibitory nutrient remains at a concentration that supports growth of the cells in the bioreactor.

Feed rates of the brine solution and second growth medium will determine the dilution rate of the inhibitory nutritional component. In one embodiment the feed rate of the brine solution relative to the feed rate of the second growth medium is greater than 20 to 1. In another embodiment the feed rate of the brine solution relative to the feed rate of the second growth medium is greater than 150 to 1. In yet another embodiment the feed rate of the brine solution relative to the feed rate of the second growth medium is greater than 300 to 1. For example, a second growth medium can be diluted with a brine solution containing 70 parts per thousand salt, at flow rates that correspond to a 328-fold dilution of the inhibitory nutritional component.

Contamination is controlled in the third feed line, feed pump, and mixing section by limiting the residence time for the growth medium mixture in the third feed line. By limiting the residence time, any cells that do enter the third feed line, feed pump, and mixing section cannot grow and populate the feed line, feed pump, and mixing section and further migrate to the reservoirs. In one embodiment the residence time in the third feed line, feed pump, and mixing section is less than 25% of the doubling time for the cells grown in the bioreactor. In another embodiment the residence time in the third feed line, feed pump, and mixing section is less than 10% of the doubling time for the cells grown in the bioreactor. In yet another embodiment the residence time in the third feed line, feed pump, and mixing section is less than 3% of the doubling time for the cells grown in the bioreactor.

In an embodiment the second growth medium is continuously fed into the bioreactor and the concentration of the electron acceptor, such as nitrate, and the carbon source, such as acetate, in the second growth medium in the nutrient feed reservoir is maintained following a period of time, such as 28 days, of continuous growth of cells, such as Pseudomonas stutzeri BR5311 (ATCC PTA-11283), in the bioreactor.

In another embodiment the concentration of the carbon source, lactate, in the second growth medium in the nutrient feed reservoir, is maintained following 28 days of continuous growth of Arcobacter sp. 97AE3-12 (ATCC PTA-11409) in the bioreactor.

### Cells Useful for the Present Invention

Cells, including microbes, mammalian cells or plant cells, which are useful for the current disclosure, may be used free or immobilized on inert, insoluble materials such as glass beads or calcium alginate.

### Microbial Cells

For the purposes of the current disclosure, any microbial cells (bacteria, fungi and yeasts), amenable to growth in bioreactors, that may be Gram positive or Gram negative, and comprising classes of strict aerobes, facultative aerobes, obligate anaerobes, and denitrifiers can be used. The bioreactor can comprise only one particular species or can comprise two or more species of the same genera or a combination of different genera of microbes, including with one or more species of each.

Examples of microbial cells useful for the disclosed method include, but are not limited to: Comamonas, Fusibacter, Marinobacterium, Petrotoga, Shewanella, Pseudomonas, Vibrio, Thauera, Microbulbife, Corynebacteria, Achromobacter, Acinetobacter, Arthrobacter, Bacilli, Nocardia, Vibrio, Actinomycetes, Escherichia, Salmonella, Arthrobacter, Acetobacter, Candida, Aspergilli, Saccharomyces, Zymomonas and Penicillium,

The present invention is particularly useful when cells grown in a bioreactor have properties promoting contamination of feeding medium stored in a connected reservoir. Properties of cells including motility and formation of biofilms may promote back growth into feeding lines and medium reservoirs connected to a bioreactor in which the cells are grown.

In particular, cells that are useful for microbially enhanced oil recovery (MEOR) may have one or both of these properties. The present bioreactor system and method may be used to grow cells for use in MEOR processes, an example of which is described in commonly owned and co-pending US Patent Application Publication #2011/0030956, which is herein incorporated by reference. The grown cells are used as an inoculum that is introduced into an oil reservoir to enhance secondary oil recovery.

In one embodiment Pseudomonas aeruginosa cells can be used in the bioreactor. In another embodiment Shewanella putrefaciens LH4:18 (ATCC PTA-8822) cells can be used in the bioreactor. In another embodiment Pseudomonas stutzeri LH4:15 (ATCC PTA-8823) cells can be used in the bioreactor. In another embodiment Pseudomonas stutzeri strain BR5311 (ATCC PTA-11283) cells can be used in the bioreactor. In another embodiment Arcobacter sp. strain 97AE3-12 (ATCC PTA-11409) or strain 97AE3-3 (ATCC PTA-11410) cells can be used in the bioreactor. In another embodiment Thauera aromatica (ATCC PTA-9497) cells can be used in the bioreactor.

Techniques and various suitable media for growth and maintenance of aerobic and anaerobic microbial cells are well known in the art and have been described in “Manual of Industrial Microbiology and Biotechnology” (A. L. Demain and N. A. Solomon, ASM Press, Washington, D.C., 1986) and “Isolation of Biotechnological Organisms from Nature”, (Labeda, D. P. ed. p 117-140, McGraw-Hill Publishers, 1990).

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and make various changes and modifications to the invention to adapt it to various uses and conditions.

### GENERAL METHODS AND APPARATUS

Optical density was measured using a Beckman Coulter DU 7500 with a one centimeter path length cell as is well known in the art.

Miller's Lauria Bertani (LB) growth medium was purchased from Mediatech (Manassas, Va.).

### Growth Media

Inoculum Growth Medium for Pseudomonas stutzeri

The filter-sterilized inoculum growth medium for Pseudomonas stutzeri had the following composition: tap water, 495 milliliter (ml); ammonium lactate, 2.5 ml of a 20 percent weight for weight (% w/w) solution; yeast extract, 0.05 grams (g); sodium nitrate, 1.0 g; NaCl, 5 g; NH₄Cl, 0.05 g; KH₂PO₄, 0.01 g; pH 6.5, filled to 500 ml with tap water.

### Inoculur Growth Medium for Arpobacter Sp

The inoculum growth medium for Arcobacter sp. was composed of tap water, 935 ml; NaCl 60 g; sodium citrate, 1.0 g; disodium fumarate, 3725 g; NH₄Cl, 100 milligram (mg); KH₂ PO₄, 50 mg; yeast extract, 500 mg; CaCl₂*2H₂O, 7.0 g; and was filter-sterilized prior to inoculation.

### First Growth Medium

The brine solution for both Pseudomonas stutzeri and Arcobacter sp. contained 70 parts per thousand (ppt) salt and was obtained from a salty aquifer in Canada.

### Second Growth Medium for Pseudomonas Stutzeri

The second growth medium, containing inhibitory nutrient components, had the following composition:

10 g of filter sterilized tap water was used to dissolve: NaAcetate, 1.53 g; NaNO₃, 3.0 g; Na₃ (PO₄), 0.16 g; NH₄Cl 0.26 g; yeast extract, 0.13 g; and the pH was adjusted to 6.5. The concentration of nitrate is inhibitory, due to nitrate concentration becoming inhibitory at greater than 1 wt %. In addition, the concentration of acetate is inhibitory, due to acetate concentration becoming inhibitory at greater than 1%.

### Second Growth Medium for Arcobacter Sp

The second growth medium, containing an inhibitory nutrient component, contained: 200 ml of filter-sterilized tap water; sodium lactate, 7.2 g; NaNO₃, 14.2 g; ammonium chloride, 720 mg; potassium dihydrogen phosphate, 144 mg; yeast extract, 3.6 g; and the pH was adjusted to 6.5. The concentration of nitrate is inhibitory, due to nitrate concentration becoming inhibitory at greater than 1 wt %. In addition, the concentration of lactate is inhibitory, due to lactate concentration becoming inhibitory at greater than 1%.

### Ion Chromatography

To quantitate nitrate, nitrite, acetate and lactate ions in aqueous media, a DX-120 chromatography unit (Dionex, Banockburn, Ill.) was used. Ion exchange was accomplished on an AS14A anion exchange column using an isocratic mixture of 3.5 millimolar (mM) Carbonate/1 mM Bicarbonate. Standard curves using known amounts of sodium nitrite, sodium nitrate, sodium lactate or sodium acetate solutions were generated and used for calibrating nitrate, nitrite, lactate and acetate concentrations.

### Example 1

### Contamination Prevention of the Nutrient Feed Reservoir During Continuous Growth of Pseudomonas Stutzeri in a Bioreactor

A multiple feed line bioreactor system (FIG. 1) was set up for continuous growth of Pseudomonas stutzeri strain BR5311 (ATCC PTA-11283) under anaerobic conditions. The nutrient feed reservoir (2) consisted of a 3 ml sterile disposable syringe (Beckton Dickinson, Franklin Lakes, N.J.) mounted on a syringe pump (Harvard Apparatus, Holliston, Mass.) which delivered a sterile second growth medium containing inhibitory nutritional components, used for Pseudoronas stutzei (composition is above), into the second feed line (3b) which connected to the third feed line (3c) and further connected to the bioreactor (6). The bioreactor consisted of a plastic tube packed with sand as an inert support. The brine solution (above) was collected from a salty aquifer and stored in the brine reservoir (1). The brine solution was pumped continuously into the bioreactor (6) through the first feed line (3a) and the third feed line (3c). The concentration of the total dissolved salt (TDS, salinity) in the brine solution was estimated at 70 ppt using a hand-held refractometer (Model RHS 10ATC, Huake Instrument Co., Ltd., Shenzhen, China).

The starter culture (inoculum) for the bioreactor (6) inoculation was prepared using a frozen inoculum (0.1 ml) of Pseudomonas stutzeri BR5311 (approximately 10¹⁰ cells per milliliter (cells/ml)). The cells were added to 500 ml, of the filter-sterilized inoculum growth medium (composition above). The inoculated medium was incubated at approximately 23° C. overnight to allow growth. The bioreactor (6) was then inoculated with the Pseudomonas stutzeri inoculum thus obtained and was shut in for 5 days. At this point, the brine solution from the brine reservoir (1) and the second growth medium, containing inhibitory nutrient components, from the nutrient feed reservoir (2) were continuously fed into the bioreactor. The feed rate of the second growth medium containing inhibitory nutritional components relative to the brine solution was such that the nutrient concentration in the second growth medium was diluted by a factor of 328 before it entered the bioreactor. The cells started growing in the bioreactor and the number of cells in the bioreactor increased to about 10⁶-10⁷ cells/ml. Periodic testing (checking growth on LB agar plates and determining the most probable numbers count known to those skilled in the art) showed that this population was maintained over the course of 28 days that the bioreactor was operated. When the medium in the bioreactor reached the level of the bioreactor outlet, effluent flowed from the bioreactor through the exit line (7).

Pseudomonas stutzei is a motile organism, which also forms biofilms. Either property, motility or biofilm formation, would have been expected to allow back growth of the cells up the feed lines and into the nutrient feed reservoir over the extended period of the bioreactor operation. However, visual inspection of the walls of the nutrient feed reservoir were clean, i.e. at the end of the bioreactor operation, i.e., 28 days, no biofilm was observed in the nutrient feed reservoir.

Results of analyses of the weight percent (wt %) of nitrate, nitrite and the growth substrate (acetate) present in the second growth medium in the nutrient feed reservoir (2) and the bioreactor effluent from the bioreactor outlet (7) are shown in Table 1 at day 0 and after 21 and 28 days from the start of the experiment. The slight differences between the concentrations of nitrate and acetate in the nutrient feed reservoir at day 0 and at days 21 and 28 are within the experimental error of the analysis. There was no visual evidence of cell growth (e.g., turbidity) in the nutrient feed reservoir (2) containing the second growth medium. In addition, no decrease in the concentration of nitrate and acetate from this growth medium in the nutrient feed reservoir was observed. If there were any contamination in the nutrient feed reservoir, a decrease in the concentration of nitrate and acetate would have been observed. However, once the second growth medium was diluted in the feed line (3c), and reached the bioreactor (6), all of the carbon source and most of the nitrate was consumed by Pseudomonas stutzeri cells in the bioreactor.

Thus application of the second growth medium containing the inhibitory nutritional components prevented cells growing in the bioreactor from back growth in feed line (3c) and contaminating the feed line and the nutrient feed reservoir.

TABLE 1
Concentration of carbon source and nitrate in the second growth medium
used in Example 1

| | | | | | Carbon | |
|---|---|---|---|---|---|---|
| | | | | | source | |
| | Nitrate | | Nitrite | | Acetate | |
| In nutrient feed | *15.1 | wt % | 0 | wt % | 7.5 | wt % |
| reservoir, (2) at | | | | | | |
| day 0 (calculated | | | | | | |
| by weight) | | | | | | |
| In nutrient feed | 15.3 | wt % | 0 | wt % | 6.7 | wt % |
| reservoir, (2) | | | | | | |
| after 21 days of | | | | | | |
| feeding | | | | | | |
| In bioreactor | 34 | ppm | 60 | ppm | 0 | ppm |
| effluent (7) after | | | | | | |
| 22 days of | | | | | | |
| feeding | | | | | | |
| In nutrient feed | 15.3 | wt % | 0 | wt % | 6.7 | wt % |
| reservoir, (2) | | | | | | |
| after 28 days of | | | | | | |
| feeding | | | | | | |
| In bioreactor | 34 | ppm | 4 | ppm | 0 | ppm |
| effluent (7) after | | | | | | |
| 28 days of | | | | | | |
| feeding | | | | | | |

*1 wt % is equel to 10,000 ppm

### Example 2

### Contamination Prevention of the Nutrient Feed Reservoir During Continuous Growth of Arcobacter Sp. in a Bioreactor

The bioreactor of Example 1 was used for growing cells of Arcobacter sp. strain 97AE3-12.

The starter culture of Arcobacter sp. for bioreactor inoculation, was prepared using colonies grown up in marine broth agar plates (HiMedia Labatories, India). A 10 microliter loop full of the colony growth was used to inoculate 20 ml of this medium and the culture was incubated aerobically at approximately 23° C. for 48 h in a 125 ml capacity flask under aerobic conditions. A diluent of this culture containing approximately 5×10⁷ cell/ml of Arcobacter sp. was then prepared to inoculate the bioreactor (6). The bioreactor was shut in for 7 days. At this point, a regimen of the brine solution and the second growth medium was continuously fed into the bioreactor. The concentration of nitrate and the carbon source (lactate) in the second medium used in this Example (Table 2) was ⅓ of that used in Example 1 (Table 1). Following growth, the number of Arcobacter sp. cells in the bioreactor increased to about 10⁶-10⁷ cells/ml; and periodic testing (checking growth on LB agar plates and most probable numbers count) showed that this population was maintained over the course of 26 days of the bioreactor operation.

Arcobacter sp. is a motile organism, which also forms biofilms. Either property, motility or biofilm formation, would have been expected to allow back growth of the cells up the feed line and into the nutrient feed reservoir over the extended period of the bioreactor operation. However, inspection of the walls of the nutrient feed reservoir at the end of the experiment showed no visible signs of biofilm formation in the nutrient feed reservoir after 26 days of the bioreactor operation.

The concentration of nitrate, nitrite and lactate in the second growth medium used for Arcobacter sp. in this Example is shown in Table 2 at day 0 and after 20 and 26 days from the start of the experiment. Continuous feeding of the second growth medium, which was diluted by the brine solution before reaching the bioreactor, resulted in consumption of both carbon source (lactate) and most of the nitrate in the bioreactor as shown in Table 2. After 20 days, tests performed as described in Example 1, did not show any evidence of cell growth (turbidity) in the nutrient feed reservoir (2). However, some nitrite was detected in the nutrient feed reservoir, indicating back growth of some of Arocbacter sp. cells from the bioreactor into the nutrient feed reservoir that resulted in consumption of some of the nitrate and its conversion to nitrite in the nutrient feed reservoir. These results suggest that the concentration of nitrate and the carbon source, lactate, in the second medium used in this Example, were not high enough to completely prevent cell back growth in the third and second feed line and resulted in some contamination of the nutrient feed reservoir (2) by further penetration of the cells from the third and second feed lines into the nutrient feed reservoir.

It can therefore be concluded that although the concentration of nutrient components in the second growth medium in this Example were lower than those used in Example 2 and did not completely prevent back growth of cells from the bioreactor into the nutrient feed reservoir, these concentrations were still high enough to prevent massive cell growth and biofilm formation in the nutrient feed reservoir.

TABLE 2
Concentration of carbon source and nitrate in the second growth medium
used in Example 2

| | | | Carbon source |
|---|---|---|---|
| | Nitrate | Nitrite | Lactate |
| In nutrient feed | 4.9 wt % | 0 wt % | 2.7 wt % |
| reservoir (2) at | | | |
| day 0 (calculated | | | |
| by weight) | | | |
| In nutrient feed | 4.4 wt % | 0 wt % | 3.2 wt % |
| reservoir, (2) | | | |
| after 20 days of | | | |
| feeding | | | |
| In bioreactor | 0 ppm | 0 ppm | 0 ppm |
| effluent (7) after | | | |
| 20 days of | | | |
| feeding | | | |
| In nutrient feed | 4.1 wt % | 1042 ppm | 3.0 wt % |
| reservoir (2) after | | | |
| 26 days of | | | |
| feeding | | | |
| In bioreactor | 13 ppm | 3 ppm | 0 ppm |
| effluent (7) after | | | |
| 26 days of | | | |
| feeding | | | |

What is claimed is:

1. A method for preventing contamination of feed lines and feed reservoirs in a bioreactor system, wherein the bioreactor system comprises a bioreactor, a brine feed reservoir, at least one nutrient feed reservoir, and at least first, second, and third feed lines that connect the feed reservoirs to the bioreactor, wherein the method comprises the steps of:
a) providing the bioreactor with an initial charge of cells and inoculum growth medium;
b) filling the first feed line with brine solution from the brine feed reservoir;
c) filling the second feed line with a second growth medium, comprising one or more inhibitory nutritional component, from the
nutrient feed reservoir; and
d) feeding the brine solution in the first feed line of (b) and the second growth medium in the second feed line of (c) into the third feed line where they combine forming a growth medium mixture;
whereby the inhibitory nutritional component of the second growth medium is diluted with brine solution in the growth medium mixture before or at the point of entering the bioreactor.

2. The method of claim 1 wherein the second growth medium containing one or more inhibitory nutritional components prevents contamination of the second feed line and the nutrient feed reservoir.

3. The method of claim 1 wherein the concentration in the growth medium mixture of the inhibitory component of the second growth medium is less than about 10% of the concentration of the inhibitory nutritional component in the second growth medium.

4. The method of claim 1 wherein the cells of (a) are cells having properties of motiliy, biofilm formation, or both motiliy and biofilm formation.

5. The method of claim 1 wherein the cells of (a) are Pseudomonas stutzeri BR5311 (ATCC PTA-11283).

6. The method of claim 1 wherein the cells of (a) are Arcobater sp. strain 97AE3-12 (ATCC PTA-11409).

7. The method of claim 1 wherein feed rate of the brine solution relative to feed rate of the second growth medium into the third feed line is greater than 20 to one.

8. The method of claim 7 wherein feed rate of the brine solution relative to feed rate of the second growth medium into the third feed line is greater than 150 to one.

9. The method of claim 8 wherein feed rate of the brine solution relative to feed rate of the second growth medium into the bioreactor is greater than 300 to one.

10. The method of claim 1 wherein the second growth medium has an acetate concentration that is greater than 1%.

11. The method of claim 10 wherein the acetate concentration in the second growth medium in the nutrient feed reservoir remains constant.

12. The method of claim 1 wherein the second growth medium has a lactate concentration that is greater than 1%.

13. The method of claim 11 wherein the lactate concentration in the second growth medium in the nutrient feed reservoir remains constant.

14. The method of claim 1 wherein the second growth medium has a nitrate concentration that is greater than 1%.

15. The method of claim 15 wherein the nitrate concentration in the second growth medium in the nutrient feed reservoir remains constant.

16. The method of claim 1 wherein the bioreactor system lacks any contamination preventing intervening physical devices in either the nutrient feed reservoir or the feed line.

17. The method of claim 1 wherein residence time of combined brine solution and second growth medium in the third feed line is less than 25% of doubling time of the cells of (a).

18. The method of claim 17 wherein residence time of combined brine solution and second growth medium in the third feed line is less than 10% of doubling time of the cells of (a).

19. The method of claim 18 wherein residence time of combined brine solution and second growth medium in the third feed line is less than 3% of doubling time of the cells of (a).

20. The method of claim 1 wherein the brine solution comprises a salt concentration that is less than about 10 weight percent.

21. A bioreactor system comprising a bioreactor, a brine feed reservoir, at least one nutrient feed reservoir containing a second growth medium comprising one or more inhibitory nutritional component, and at least first, second, and third feed lines that connect the feed reservoirs to the bioreactor, wherein the first and second feed lines each connect to a reservoir at one end and to the third feed line at the other end.
